(19) United States
(12) Patent Application Publication
(10) Pub. No.: US 2017/0326348 A1
(43) Pub. Date: Nov. 16, 2017

(54) SURGICAL DRAIN MANAGEMENT APPARATUS

(71) Applicant: iInspire, LLC, Georgetown, KY (US)

(72) Inventors: Jennifer Baker, Georgetown, KY (US); Marvin Baker, Georgetown, KY (US)

(73) Assignee: iInspire, LLC, Georgetown, KY (US)

(21) Appl. No.: 15/595,883

(22) Filed: May 15, 2017

Related U.S. Application Data
(60) Provisional application No. 62/335,682, filed on May 13, 2016.

(51) Int. Cl. A61M 39/08; A45F 5/02; A61M 27/00

(52) U.S. Cl. CPC A61M 39/08; A61M 27/00; A45F 5/021; A61M 2209/088

(57) ABSTRACT

A post-surgical drain management pouch is disclosed. The pouch has resilient tubing clamps to hold the tubing in a friction fit arrangement. The guides organize drain tubing and prevent their binding while also providing sufficient slack to prevent the tubing from dislodging or otherwise accidentally discharging the drainage from a surgical site. The clips also prevent the accidental disconnection of the drain tubing from the drain if the tubing is pulled tight.

### I. RELATED APPLICATIONS

This application claims the benefit of U.S. Provisional Patent Application 62/335,682 filed on May 13, 2016.

### II. TECHNICAL FIELD

This application discloses claims and embodiments regarding the management of surgical drains utilizing a wearable apparatus.

### III. BACKGROUND

A Jackson-Pratt (JP) drain is a type of post-operative closed-suction medical device that is placed in an incision during surgery for the purpose of facilitating the removal of blood and interstitial fluid from surgical sites. The drain is comprised of a hollow tube that is connected to an egg-shaped bulb. The hollow tube begins inside the incision and exits the body. Attached to the end of the tube outside of the body is the collection bulb which is depressed and released to create a vacuum in the bulb as compared to the tubing, thus creating suction. The purpose of the JP drain is to promote healing while preventing infection.

Typically, the tubing and bulb are either free-hanging or affixed to the body by tape. Unfortunately, the fluid and tissue collected by the JP drain are unsightly. Moreover, visible tubing and/or collection bulbs, and their contents, can cause great embarrassment for the patient.

Two problems commonly arise when dealing with JP drains. First, for at least some period during the few days or weeks the drains are installed, the patient is ambulatory and storing the drains and tubing when the patient moves about is a common problem. Second, the unsightliness of bulbs and tubing, and especially their contents, can lead to non-compliance in public and a decrease in ambulation and therefore slower healing.

As to the securing the drains, medical personnel often pin the drains to the inside of the patient's hospital gown. This works poorly at best because the gown is not attached to the patient and thus the drains may become displaced from the area of the surgical wound potentially unseating the drains as the patient moves about. This is particularly problematic when the patient is bedridden and turns in bed. When the patient is ambulatory, the simple act of bathing or using the toilet becomes a difficult chore because care must be used to ensure that the drains are not significantly moved when lifting the patient's gown and prematurely removed from the incision.

Because of these shortcomings, alternate means of securing the drains have been used. For example, surgical tape may be used to physically tape the drain to the patient, but this is uncomfortable and renders it difficult to empty the drain without first un-securing it. Various devices have been disclosed to deal with this problem. For example, U.S. Pat. No. 6,273,872 discloses a belt used to support, among other things, vacuum surgical drains. The principle difficulty with this device is that the drains are exposed and thus prone to becoming entangled in clothing as well as being unsightly. D311,453 discloses a generic bag to be worn about the body which obviously may be employed to hold surgical drains. However, none of these devices provide means of organizing multiple surgical drains. After discharge from the hospital, patients themselves suffer from the same problems and have devised a number of impromptu methods of dealing with JP drains.

Thus, there continues to be a need for improvements in the management of medical tubing and bulb(s) used in the drainage of interstitial and other waste fluids from an incisional locus.

### IV. SUMMARY

This application presents claims and embodiments that fulfill a need or needs not yet satisfied by existing products. The applicant is unaware of any product, device, method, disclosure or reference that discloses the features of the claims and embodiments disclosed herein, and as more fully described below.

In one embodiment of the disclosure, a post-surgical pouch comprises a plurality of walls defining an interior space, a plurality of tubing guides disposed within the interior space of the walls, and means for securing the pouch to a user.

In another embodiment of the disclosure, a post-surgical pouch comprising a plurality of walls defining an interior space, a plurality of tubing guides disposed within the interior space of the walls, a slit formed in one of the walls, and means for securing the pouch to a user.

In another embodiment of the disclosure, a post-surgical pouch comprising a pouch having a plurality of walls having top, bottom, right side, and left side members and further possessing an interior surface and an exterior surface, means for sealing the pouch, a plurality of inlets comprising slits in the walls of the pouch, means for retaining medical tubing, the tubing retention means affixed to the interior surface of the walls, and means for retaining the pouch, the pouch retention means disposed on the body of the wearer.

It is envisioned that each of the embodiments described above may further include the following combination and sub-combination of additional elements.

For example, in one or more embodiments, the walls comprise a base and a top having an exterior surface and defining the interior space. The interior space is bound by an interior surface opposing the exterior surface of the base and top of the walls. The walls may further comprise flexible material. The flexible material allows a user to manipulate any internally placed articles, including suction bulbs in fluid communication with the tubing inserted through the pouch. The walls may further comprise antibacterial material to reduce, inhibit, or prevent the promotion or growth of bacterial agents.

In another example, the pouch further comprises a cover disposed over the interior space of the walls. The cover encloses the contents of the interior space to prevent unnecessary ingress or egress of unwanted items or materials.

In another example, the pouch further comprises means for closing access to the interior space of the walls. Closure means securely entrains or entraps the contents of the interior space to prevent unwanted or accidental dislodging of the contents therein.

In another example, each one of the plurality of tubing guides comprises an arcuate recess bound by a pair of opposing detents. Each one of the opposing detents comprises returnably resilient material having an inward bias. The detents retain the tubing therewith. The detents are inwardly biased to prevent the tubing from easily working-free from the recess(es).

In another example, the pouch further comprises a first slit and a second slit. The first slit and second slit are mutually disposed in opposing corners of the interior space.

In another example, securing means comprises an adjustable strap. In another example, securing means comprises an adjustable belt. It is further envisioned that securing means comprises a combination of adjustable strap and adjustable belt.

### V. BRIEF DESCRIPTION OF THE DRAWING(S)

The advantages and features of the present invention will become better understood with reference to the following more detailed description and claims taken in conjunction with the accompanying drawings, in which like elements are identified with like symbols, and in which:

FIG. 1 is a front cutaway elevation view of a pouch.

FIG. 2 is a partial front view of FIG. 1 with the interior surface and interior space exposed.

FIG. 3 is a front elevation view of the pouch.

### VI. DETAILED DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments of pet article, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

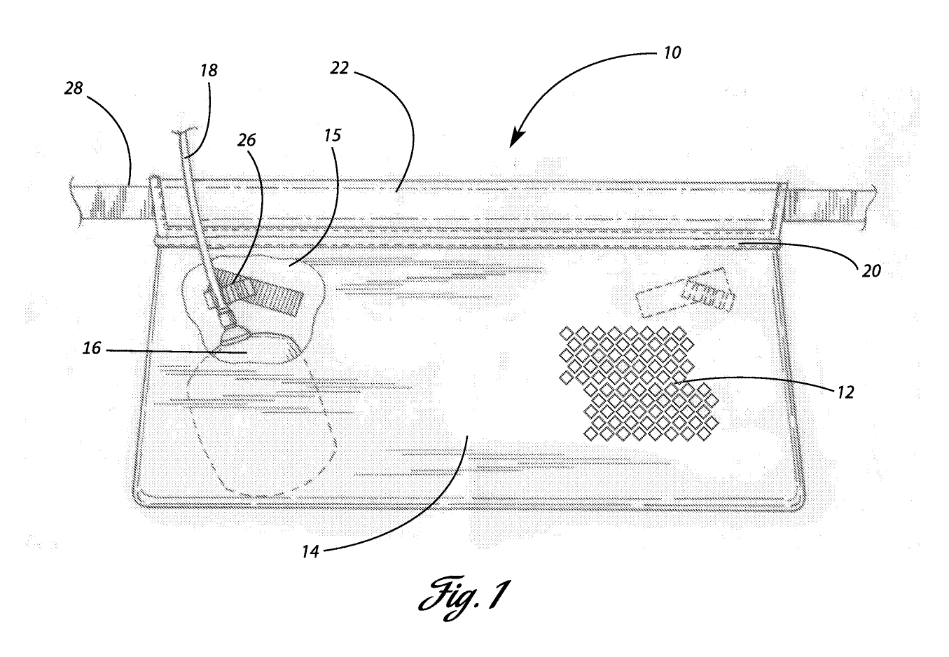

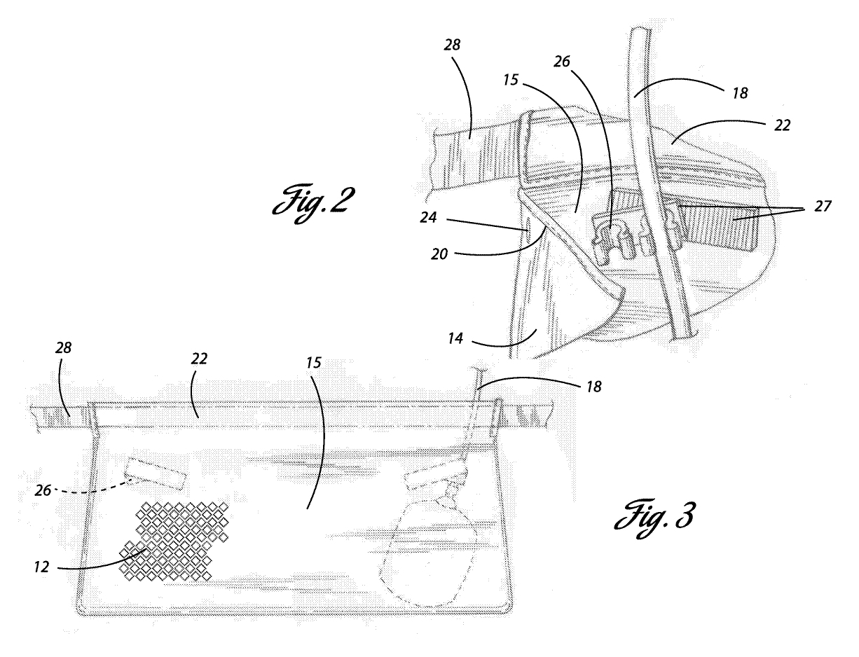

In accordance with the figures appended hereto, a device 10 is depicted in FIGS. 1-3 comprising a pouch 12 that provides means for management and storage of surgical drains 16, i.e. bulbs, and tubing 18. The pouch 12 is preferably comprised of a plurality of walls 14 having a base, a top, an interior surface, and an exterior surface. The walls 14 surround an interior compartment or space 15 large enough to house a plurality of drains 16.

The tubing 18 leads down to a pouch 12 worn typically about the patient's waist. The pouch 12 is worn at a point on the body as to permit some slack in the tubing 18 to facilitate movement without pulling the tubing 18 taught between the drain 16 and incision. In one embodiment, the bases of the walls 14 are affixed to a floor having a top surface and a bottom surface. In another embodiment, the base of the walls 14 are affixed to each other to form the bottom of the pouch 12.

The pouch 12 is reversibly sealed along the top of the walls 14 by a zipper, loop and hook fabric, snap, or similar fastening means 20. In an embodiment, a cover, lid or flap 22 seals the pouch 12 along the tops of the walls 14.

It is also envisioned that a slit 24 may be provided in at least one wall 14 that permits the entry of the tubing 18 into the pouch 12. In an embodiment, a plurality of slits 24 are provided and positioned at the top rear corners of the pouch 12. In an embodiment, the pouch 12 is constructed from a washable mesh so as to allow the device 10 to be worn in the shower. In an alternative embodiment, the interior of the pouch 12 is capable of retaining liquids and thus inhibit leaks from the pouch 12. The pouch 12 is also pliable so as to allow the bulbs 16 to be compressed within the pouch 12 by applying pressure to an exterior surface of a wall 14 of the pouch 12. The pouch 12 may be worn with a strap 28 over the shoulder or around the waist. In a preferred embodiment, the straps 28 are adjustable to different lengths. In the event of surgical drains 16 being utilized on both sides of the body, the pouch 12 would be worn in the center of the midsection. In a yet further embodiment, the material from which the pouch 12 is made has antibacterial properties so as to be antiseptic.

At least one, and preferably a plurality of tubing clips 26 are removably affixed to the interior surfaces of the walls 14 of the pouch 12 to secure the tubing 18 to the walls 14. The tubing clips 26 are preferably arcuate clips adapted and configured to retain the tubing 18 in a friction fit arrangement. The arms or prongs of the clips may be forced open to accept the tubing 18 into an arcuate orifice but are inwardly biased or tensioned and/or comprised of a returnably resilient material so as to close back around the tubing 18 once the tubing 18 is inserted or removed. Notably, the clips 26 do not restrict the free flow of liquids to the bulb 16 but merely surround the exterior of the tubing 18 to secure it within the pouch 12. Clip fasteners 27, e.g. magnets or hook and loop fabric, may be employed to removably affix the clips to the interior surface of the wall 14 within the pouch 12. The clips 26, being made of a resilient material, are preferably removed if the pouch 12 is to be washed so as to prevent a deterioration of their resiliency. Alternatively, the clips 26 are permanently affixed to the walls 14. Nothing within this disclosure is intended to limit the device 10 or clips 26 to the disclosures herein.

The tubing 18 section is secured near the bulb 16 and bulb discharge port and serves to secure the bulb 16 within the pouch 12 and against the interior surface of a wall 14. Securing the tubing 18 and storing the bulbs 16 in a manner that permits some slack in the tubing 18 while supporting the bulb 16 while permitting a greater freedom of movement of the patient, thus increasing ambulation. Securing the tubing 18 in a friction fit arrangement inhibits the detachment of the tubing 18 from the bulb when the tubing 18 is pulled tight, thus preventing the spilling of body fluids contained within the drain that could present a biohazard.

It is envisioned that the various embodiments, as separately disclosed, are interchangeable in various aspects, so that elements of one embodiment may be incorporated into one or more of the other embodiments, and that specific positioning of individual elements may necessitate other arrangements not specifically disclosed to accommodate performance requirements or spatial considerations.

It is to be understood that the embodiments and claims are not limited in its application to the details of construction and arrangement of the components set forth in the description and illustrated in the drawings. Rather, the description and the drawings provide examples of the embodiments envisioned, but the claims are limited to the specific embodiments. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the U.S. Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. It is intended that the application is defined by the claims appended hereto.

What is claimed is:

1. A post-surgical drain management pouch comprising:
a plurality of walls defining an interior space having a closed base and sides, and a resealable opening at the top of said walls; at least one resilient tubing clip disposed within said interior space of the walls; and at least one of a belt or strap to secure said pouch to said user.

2. The pouch of claim 1, wherein said tubing clips are constructed of a resilient material.

3. The pouch of claim 1, wherein said tubing clips are removably attached.

4. A post-surgical management pouch comprising:
a plurality of walls defining an interior space having a closed base and sides, and an opening at the top of said walls;
at least one tubing clips disposed within the interior space of the walls;
at least one slit formed in said walls through which drain tubing may pass; and
at least one of a strap or belt to secure said pouch to a user.

5. The pouch of claim 4, wherein said tubing clip retains said drain tubing in a friction fit arrangement.

6. The pouch of claim 5 further comprising a cover disposed over the top of said walls.

7. The pouch of claim 6 further comprising a fastening means for reversibly closing said pouch along the tops of said walls.

8. The pouch of claim 4, wherein said walls are constructed of a mesh fabric.

9. The pouch of claim 4, wherein said walls comprise antibacterial material.

10. The pouch of claim 4, wherein each said tubing clip comprises an arcuate recess bound by a pair of opposing detents.

11. The pouch of claim 7, wherein each one of the opposing detents comprises returnably resilient material having an inward bias.

12. The pouch of claim 4, further comprising a first slit and a second slit, said first slit and said second slit mutually disposed in opposing corners of said interior space.

13. The pouch of claim 4, wherein said securing means comprises an adjustable strap.

14. The pouch of claim 4, wherein said securing means comprises an adjustable belt.

15. A post-surgical pouch comprising:
a. a pouch having a plurality of walls having top, bottom, and side members, said pouch having a closed base and sides, an opening at the top of said walls, and further possessing an interior surface and an exterior surface;
b. fastening means to reversibly close said opening;
c. a plurality of inlets comprising slits in said walls of the pouch;
d. at least one tubing clip to retain drain tubing, said clip affixed to said interior surface of said walls; and
e. means for retaining said pouch, said pouch retention means disposed on the body of the wearer.

16. The pouch of claim 15, wherein tubing retention means comprises an arcuate recess bound by a pair of opposing detents.

17. The pouch of claim 16, wherein each one of said opposing detents comprises returnably resilient material having an inward bias.

18. The pouch of claim 15, where said clips are removably affixed to the pouch.

19. The pouch of claim 15, wherein said pouch retention means comprises an adjustable strap.

20. The pouch of claim 15, wherein pouch retention means comprises an adjustable belt.
